(19) United States
(12) Patent Application Publication
(10) Pub. No.: US 2026/0124066 A1
(43) Pub. Date: May 7, 2026

(54) ACOUSTIC SILENCER FOR A URINE SUCTION SYSTEM

(71) Applicant: PureWick Corporation, Covington, GA (US)

(72) Inventors: Tony Garvey, Listowel (IE); Meghan Caracciola, Washington, DC (US); Padraig Fogarty, Ballina-Killaloe (IE); Tamika Richardson, Schenectady, NY (US)

(21) Appl. No.: 19/353,151

(22) Filed: Oct. 8, 2025

Related U.S. Application Data
(63) Continuation of application No. 17/653,920, filed on Mar. 8, 2022, now Pat. No. 12,458,525.
(60) Provisional application No. 63/159,210, filed on Mar. 10, 2021.

(51) Int. Cl. A61F 5/453; F04B 39/00

(52) U.S. Cl. CPC A61F 5/453; F04B 39/0061; F04B 39/0066; F04B 39/0072

(57) ABSTRACT

An example urine suction device suitable for extracting urine from a urine collection device is disclosed. The suction device can include a motor, a pump driven by the motor, a conduit, and an acoustic silencer. The pump can extract urine from a urine collection device. The acoustic silencer can be in fluid communication with the pump and conduit. The acoustic silencer can attenuate or reduce one or more acoustic waves generated by the pump and/or the motor. One or more components or structures within the acoustic silencer can at least partially attenuate the acoustic waves as they propagate along a non-linear airflow path defined by the acoustic silencer.

### CROSS-REFERENCE TO RELATED APPLICATION(S)

This application is a continuation of U.S. Application No. Ser. No. 17/653,920 filed on Mar. 8, 2022, which claims priority to U.S. Provisional Patent No. 63/159,210 filed on Mar. 10, 2021, the disclosure of each of which is incorporated herein, in its entirety, by this reference.

### BACKGROUND

In various circumstances, a person may have limited or impaired mobility such that typical urination processes are challenging or impossible. For example, a person may experience or have a disability that impairs mobility. A person may have restricted travel conditions such as those experienced by pilots, drivers, and workers in hazardous areas. Additionally, sometimes urine collection is needed for monitoring purposes or clinical testing.

In these and other circumstances, a urine collection device can be utilized to collect and retain evacuated urine. After the urine has been evacuated, the urine may need to be removed from the urine collection device. For example, a urine suction device can be used to remove urine evacuated into the urine collection device.

Devices, systems, and methods which more efficiently, hygienically, and discreetly remove or otherwise transport urine can be desirable.

### SUMMARY

Embodiments are directed to a urine suction device including a motor, a pump, a conduit, and an acoustic silencer. The pump is configured to be driven by the motor. The pump is configured to extract urine from a urine collection device. The conduit is coupled to the pump and configured to exhaust a fluid from the pump. The acoustic silencer is in fluid communication with the conduit and the pump. The acoustic silencer defines a non-linear airflow path between an inlet and an outlet of the acoustic silencer.

In another embodiment of the present disclosure, a system for transporting urine away from a user is disclosed. The system can include a urine collection device, a container, and a suction device coupled to the urine collection device. The suction device is configured to draw urine from the urine collection device into the container. The urine suction device can include a motor, a pump, a conduit, and an acoustic silencer. The pump is configured to be driven by the motor and extract urine from the urine collection device. The conduit is coupled to the pump and configured to exhaust a fluid from the pump. The acoustic silencer is in fluid communication with the conduit and the pump. The acoustic silencer defines a non-linear airflow path between an inlet and an outlet of the acoustic silencer.

Embodiments are directed to a method for reducing noise pollution of a urine suction device with an acoustic silencer. While power is being supplied (e.g., electrical power, pneumatic power, etc.), the motor can actuate a pump of the urine suction device to transport urine away from the user. The method also includes routing one or more acoustic waves generated by the pump to the acoustic silencer through a conduit. The method also includes providing the one or more acoustic waves at an inlet of the acoustic silencer. The method can also include attenuating at least a portion of the one or more acoustic waves along an airflow path formed within the acoustic silencer. The method can also include releasing the one or more acoustic waves from an outlet of the acoustic silencer.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 is a block diagram of a urine suction device coupled to a urine collection device, according to an embodiment.

FIG. 2A is an isometric view of a urine suction device and a urine collection device operably associated with a user, according to an embodiment.

FIG. 2B is an isometric view of a urine suction device and a urine collection device operably associated with a user, according to an embodiment.

FIG. 3 is a schematic cross-section view of a urine suction device, according to an embodiment.

FIG. 4 is a schematic cross-section view of an acoustic silencer for a urine suction device, according to an embodiment.

FIG. 5 is a schematic cross-section view of an acoustic silencer for a urine suction device, according to another embodiment.

FIG. 6 is a schematic cross-section view of an acoustic silencer for a urine suction device, according to another embodiment.

FIG. 7 is an example flow diagram of a method for reducing noise pollution of a urine suction device.

### DETAILED DESCRIPTION

Embodiments described herein are directed to urine suction devices suitable for discreetly pumping urine from a urine collection device and away from the body of a person. The embodiments of the urine suction devices described herein may be fluidly coupled or otherwise attached to a urine collection device positioned in a pubic region of a user to collect urine or other fluids expelled by the user. While this disclosure primarily describes the suction device as a urine suction device, it will be appreciated that the embodiments disclosed herein can also be utilized to collect other fluids discharged from a user.

In embodiments, the urine suction device includes a housing forming or defining an interior volume. One or more components of the urine suction device can be at least partially disposed within the interior volume. For example, a motor (e.g., an electric or pneumatic motor) and a pump can be disposed within the housing and operate to provide a suction force. In some embodiments, the housing can be omitted. While the urine suction device is operating (e.g., pumping urine from a urine collection device), the pump and/or another component of the urine suction device can generate an audible noise (e.g., one or more acoustic waves). In some embodiments, the pump can include a conduit which enables air to exhaust from the pump. As pumped air is exhausted from the conduit, acoustic waves can emit from the conduit to generate audible noise. This noise can be embarrassing and unflattering to the user of the urine suction device, especially while the user in a public space or has company. As such, reducing or eliminating acoustic waves generated by the urine suction device can be desirable to provide a urine suction device that is more discreet and less obtrusive.

In some embodiments, the urine suction device can include a motor and a pump that is driven by the motor. For example, the motor can be coupled to a diaphragm within the pump which draws air into and out of the pump. While driven by the motor, the pump can generate a negative pressure that can draw urine from a urine collection device. The urine suction device can include a conduit coupled to the pump which enables fluids (e.g., air) to be exhausted from the pump. The urine suction device includes an acoustic silencer in fluid communication with the conduit and pump. For example, the acoustic silencer can include an inlet which is attached or otherwise affixed to the conduit.

The acoustic silencer can define one or more non-linear airflow path between the inlet and an outlet of the acoustic silencer to attenuate or otherwise reduce one or more acoustic waves generated by the pump and propagated through the conduit. Components and/or structures disposed within the acoustic silencer can define a plurality of non-linear airflow paths within the acoustic silencer. In other words, acoustic waves that propagate into the acoustic silencer can be directed and guided along one or more paths within the acoustic silencer which cause the acoustic waves to be attenuated or otherwise reduce the magnitude of the acoustic waves. While the non-linear airflow path is described as a singular path in some embodiments, the non-linear airflow path can include multiple paths that diverge and/or intersect relative to components and/or structures within the acoustic silencer.

In some embodiments, the acoustic silencer can include an enclosure and one or more components which attenuate acoustic waves within the enclosure. For example, the acoustic silencer can include noise dampening materials, such as, foam, mineral wool, polyester, and/or any other material known to dampen or attenuate acoustic waves. Additionally, or alternatively, the acoustic silencer can include one or more components which attenuate acoustic waves within the enclosure by redirecting the acoustic waves to generate destructive interference. In other words, one or more components or structures within the acoustic silencer can direct the acoustic waves along the non-linear airflow path which attenuates the acoustic waves as they propagate within the acoustic silencer. For example, the acoustic silencer can include multiple internal chambers, one or more conduits, one or more baffles, internal walls, plates, a combination thereof, or any other element capable of directing acoustic waves along the non-linear airflow path.

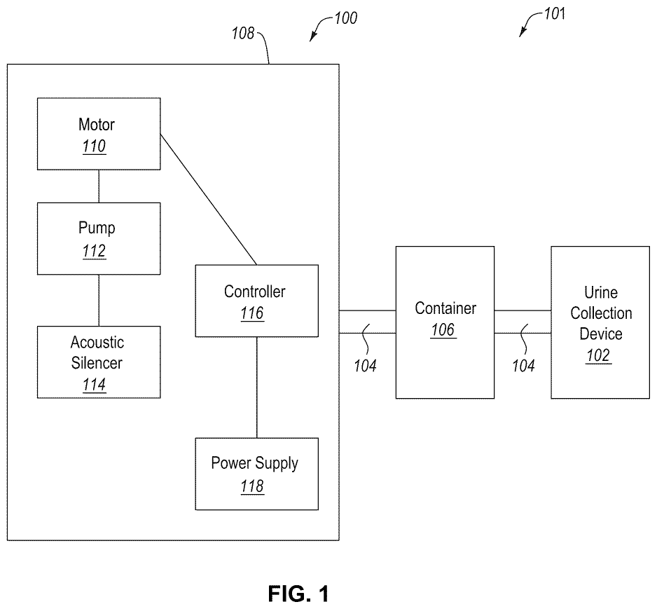

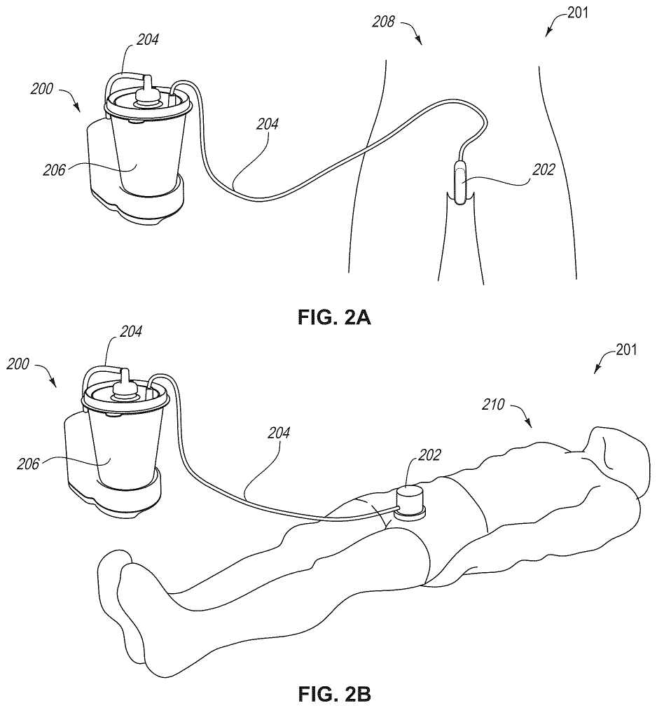

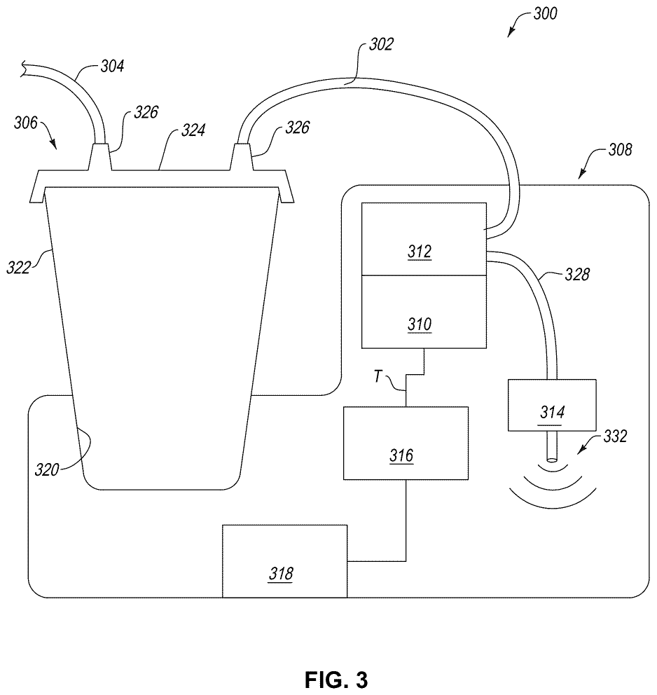

FIG. 1 shows a block diagram of a urine collection system 101 including a urine suction device 100 coupled to a urine collection device 102, according to an embodiment. The urine suction device 100 can be coupled to the urine collection device 102 using one or more tubes 104 and a container 106. For example, a suction force generated by the urine suction device 100 can draw urine or other fluids from the urine collection device 102, through the tube 104, and into the container 106. While this embodiment illustrates the urine suction device 100 and the container 106 as separate and distinct, the container 106 can be incorporated into or retained by the urine suction device 100 in other embodiments (as shown in FIGS. 2A-3).

The urine suction device 100 can include a housing 108, a motor 110, a pump 112, an acoustic silencer 114, a controller 116, and a power supply 118. The housing 108 can define an internal volume and one or more of the components of the urine suction device 100 (e.g., the motor 110, the pump 112, the acoustic silencer 114, the controller 116, the power supply 118, etc.) can be disposed within the internal volume. For example, one or more recesses or attachment features (not shown) can be molded machined or affixed to the housing 108 to provide an attachment surface for one or more of the components of the urine suction device 100. As shown in FIGS. 2A-3, the housing 108 can also form a recess or cavity for retaining the container 106. The recess or cavity can engage a base portion of the container 106 to retain the container 106 to the urine suction device 100. The housing 108 can be assembled using one or more distinct pieces that are adhered, affixed, or otherwise assembled to form the housing 108. For example, the housing 108 can be formed of one or more molded, co-molded, machined, or stamped elements. The housing 108 can be manufactured from a polymer, ceramic, metal, a combination thereof, or any other material that can provide the functionality described herein.

While not depicted in FIG. 1, the housing 108 can support one or more input devices, such as, buttons, toggles, switches, dials, touchscreens, levers, any combination thereof, or any other input device. The one or more input devices can cause the urine collection device 100 to alter its operational state, for example, by causing the controller 116 to supply electrical power to the motor 110 to generate suction via the pump 112. The housing 108 can include various through-holes, ports, or apertures which accommodate the one or more input components. Additionally, or alternatively, the housing 108 can include various through-holes or apertures which accommodate tubes, wires, or any other fluid or electrical conduit that may need to extend into the internal volume of the housing 108. For example, an electrical cable can extend from an electrical wall outlet and through an aperture within the housing 108 to the power supply 118 to electrically power the urine suction device 100. The housing 108 will be further described below with reference to FIGS. 2A-3.

In embodiments, the motor 110 can be any electrical motor or pneumatic motor capable of operating the pump 112 (i.e., causing the pump 112 to generate a negative pressure within the container 106 and/or the urine collection device 102). For example, the motor 110 can operate on alternating current (AC) or direct current (DC) and can be a brushless motor or a brushed motor. Additionally, or alternatively, the motor 110 can be DC series motor, a DC shunt motor, a stepper motor, a linear motor, a servo motor, a combination thereof, or any other motor capable of the functionality described herein. The motor 110 can drive the pump 112 to generate a suction force to draw urine out of the urine collection device 102, through the tube 104, and into the container 106. During operation (e.g., when electrical or pneumatic power is being supplied to the motor), the motor 110 can generate noise, such as, acoustic waves resultant of movement and vibration associated with operation. At least a portion of these acoustic waves generated by vibration of the motor 110 can be transferred to the pump 112. The acoustic silencer 114 can also at least partially attenuate any acoustic waves generated by the motor 110 that are transferred to the pump 112 and propagate through the conduit. The motor 110 will be further described below with reference to FIGS. 2A-3.

The pump 112 can be any pump capable of generating a suction force which can draw urine from the urine collection device 102. For example, the pump 112 can be a rotary lobe or gear pump, a diaphragm pump, a piston, a screw pump, a combination thereof, or any other pump capable of the functionality described herein. In some embodiments, the pump 112 can include a diaphragm (not shown) that draws air into the pump on a first stroke and exhausts air out of the diaphragm on a second stroke. The first and second strokes of the diaphragm can be actuated by rotation of the motor 110. During operation (e.g., when rotation of the motor causes actuation of the pump), the pump 112 can generate noise, such as, acoustic waves resultant of movement and vibration associated with operation. At least some of these acoustic waves can propagate along a conduit coupled to the pump 112. The conduit can exhaust air drawn into the pump 112, for example, on the second stroke of the diaphragm. The pump 112 will be further described below with reference to FIGS. 2A-3D.

In some embodiments, the acoustic silencer 114 can be in fluid communication with the conduit and the pump 112. For example, the conduit can be coupled to the pump 112 and the acoustic silencer 114 such that air exhausted from the pump 112 propagates through the conduit and into the acoustic silencer 114. The conduit can be coupled, adhered, fastened, molded, welded, or otherwise affixed to the pump 112 by any mechanism. The conduit can be coupled, adhered, fastened, molded, welded, or otherwise affixed to the acoustic silencer 114 by any mechanism.

In some embodiments, the acoustic silencer 114 can include noise dampening materials and/or one or more components which attenuate acoustic waves within the acoustic silencer 114 by redirecting the acoustic waves to generate destructive interference. The one or more components within the acoustic silencer 114 can direct the acoustic waves along the non-linear airflow path which attenuates the acoustic waves as they propagate within the acoustic silencer 114. For example, the acoustic silencer can include multiple internal chambers, one or more conduits, one or more baffles, internal walls, plates, a combination thereof, or any other structure capable of directing acoustic waves along the non-linear airflow path. In some embodiments, the acoustic silencer 114 can attenuate or otherwise reduce an amplitude of the acoustic waves by at least 5%, about 5% to about 15%, about 15% to about 30%, about 30% to about 50%, about 50% to about 75%, or more than 75%. The acoustic silencer 114 will be further described below with reference to FIGS. 2A-3.

In some embodiments, one or more of the components of the urine suction device 100 can be operably coupled to the controller 116. For example, the controller 116 can be disposed within the internal volume of the housing 108 and include one or more processors and memory storage having one or more operational programs stored therein. When executed by the processor, the one or more operational programs can cause the urine suction device 100 to operate (e.g., draw urine or other fluid from a urine collection device). The controller 116 can be communicatively coupled to one or more of the components of the urine suction device 100 (e.g., the motor 110, the pump 112, the power supply 118, etc.). The controller 116 will be further described below with reference to FIGS. 2A-3.

The power supply 118 may be operably coupled to the controller 116, the motor 110, the pump 112, or any other components of the urine suction device 100 to provide electrical power to the urine suction device 100. For example, the power supply 118 may include one or more batteries (e.g. lithium-ion, nickel-cadmium, nickel-metal hydride, etc.) or portable chargers (e.g., power banks). The one or more batteries may be rechargeable. In examples, the one or more batteries may be modular battery packs, which may be removed and replaced. In examples, the one or more batteries have a connection for charging, such as a connection for the portable charger. The power supply 118 may be a replaceable and rechargeable battery, such as a 12 volt battery. The rechargeable battery may be a lithium ion battery, lithium-ion polymer, a nickel-cadmium battery, nickel-metal hydride, lead acid, etc., batteries. The power supply 118 may include a plurality of rechargeable batteries. The rechargeable battery may be at least a 1 volt battery, such as 1.5 volts to 3 volts, 3 volts to 6 volts, 6 volts to 9 volts, 9 volts to 12 volts, 12 volts to 15 volts, 15 volts to 24 volts, greater than 12 volts, less than 24 volts, or less than 15 volts.

In examples, the power supply 118 may include a cord or wired connection for connecting to a power outlet. For example, the power supply 118 may include 110 volt, 220 volt, or similar connections. The cord may allow the user to plug the urine suction device 100 into a power outlet in a room, an extension cord, or a power station or power bank (e.g., battery pack or bank). Accordingly, the power supply 118 may include a wall outlet, the extension cord, or a power station or power bank. In examples, the power supply 118 may include both a wired connection for coupling to a power source and a battery pack. Accordingly, the urine suction device 100 may be run with our without battery power. In examples, the wired connection may be provided as a detachable power cord which may be removed from the urine suction device 100. The wired connection may serve to recharge the battery pack and provide power to the urine suction device 100.

The container 106 can be distinct from the urine suction device 100 or releasably retained on the housing 108 of the urine collection device 100 (as shown in FIGS. 2A-3). The container 106 can be any container capable of storing urine or other fluids that are extracted from the urine collection device 102 by a suction force applied by the urine suction device 100. As such, the container 106 can be manufactured of any rigid or flexible material capable of reliably retaining fluid. The container 106 can be machined, molded, co-molded, or otherwise manufactured from any material, such as, a polymer, a metal, a ceramic, or a combination thereof. The container 106 will be further described below with reference to FIGS. 2A-3.

The urine collection device 104 can be any urine collection device presently available or otherwise developed in the future. The urine collection device 104 may be adapted to extract urine or other fluids discharged by female and/or male persons. One suitable non-limiting example of a urine collection device that can be used is an external catheter, available from Pure Wick, Inc. Any urine collection device capable of collecting discharged urine from a male or female user can be utilized with the present disclosure. For example, the urine collection devices described in U.S. Patent Publication No. 2019/0282391 filed 6 Jun. 2019 and U.S. Pat. No. 10,390,989 filed 8 Sep. 2016, the disclosures of which are incorporated herein, in their entirety, by this reference.

FIGS. 2A and 2B show isometric views of a urine collection system 201 including a urine suction device 200 and a urine collection device 202, according to an embodiment. Except as otherwise disclosed herein system 201 (e.g., the urine suction device 200 and the urine collection device 202) may be the same or substantially similar to any of the urine collection systems disclosed herein. The urine collection system 201 includes a first tube 204 extending from the urine suction device 200 to a container 206 and a second tube 204 extending from the container 206 to the urine collection device 202. The urine collection device 202 can be disposed on a female user 208 (as shown in FIG. 2A) or a male user 210 (as shown in FIG. 2B). On female users, the urine collection device 202 can be placed between the legs or labia and held snugly against the external urethra by the pressure of friction from the user's body, by the pressure of the legs or by such means as an undergarment, elastic strips, and/or adhesive tape. Examples of the urine collection device 202 configured to be used with female users are disclosed in U.S. Pat. No. 10,973,678 filed on Jun. 2, 2017, U.S. Pat. No. 10,390,989 filed on Sep. 8, 2016, U.S. Pat. No. 10,226,376 filed on Jun. 3, 2017, PCT Patent Application No. PCT/2022/011281 filed on Jan. 5, 2022, and PCT Patent Application No. PCT/US 2020/042262 filed on Jul. 16, 2020, the disclosures of each of which is incorporated herein, in its entirety, by this reference. In male applications, the urine collection device 202 can be positioned around the penis. Examples of the urine collection 202 configured to be used with male users are disclosed in PCT Patent Application No. PCT/US2021/039866 filed on Jun. 30, 2021, U.S. patent application Ser. No. 16/433,773 filed on Jun. 6, 2019, PCT Patent Application No. PCT/2022/011281 filed on Jan. 5, 2022, and PCT Patent Application No. PCT/US 2020/042262 filed on Jul. 16, 2020, the disclosures of each of which is incorporated herein, in its entirety, by this reference. The urine suction device 200 can generate a negative pressure within the container 206 to draw or extract urine or other fluids from the urine collection device 202 into the container 206.

FIG. 3 show a non-limiting example of a urine suction device 300, according to an embodiment. FIG. 3 shows a schematic cross-section view of the urine suction device 300, according to some embodiments. The urine suction device 300 can be substantially similar to and can include some or all of the features of the urine suction devices 100 and 200. For example, one or more tubes 304 can place the urine suction device 300 in fluid communication with a container 306 and/or a urine collection device (not shown).

The urine suction device 300 can include a housing 308, a motor 310, a pump 312, an acoustic silencer 314, a controller 316, and a power supply 318. The motor 310 can be substantially similar to and can include some or all of the features of the motor 110 described with reference to FIG. 1. For example, the motor 310 can drive the pump 312 to generate a suction force to draw urine out of the urine collection device (not shown), through the tube 304, and into the container 306. The pump 312 can be substantially similar to and can include some or all of the features of the pump 112 described with reference to FIG. 1. For example, the pump 312 can be a rotary lobe or gear pump, a diaphragm pump, a piston, a screw pump, a combination thereof, or any other pump capable of the functionality described herein. The pump 312 can apply a suction force to the container 306 through a tube 302. The pump 312 and the container 306 can be in fluid communication through the tube 302. The controller 316 can be substantially similar to and can include some or all of the features of the controller 116 described with reference to FIG. 1. For example, the controller 316 can be communicatively coupled to one or more of the components of the urine suction device 300 (e.g., the motor 310, the pump 312, the power supply 318, etc.) through electrical traces T. The power supply 318 can be substantially similar to and can include some or all of the features of the power supply 118 described with reference to FIG. 1. For example, the power supply 318 may be operably coupled to the controller 316, the motor 310, the pump 312, or any other components of the urine suction device 300 to provide electrical power to the urine suction device 300.

The housing 308 can be substantially similar to and can include some or all of the features of the housing 108 described with reference to FIG. 1. While the housing 308 is illustrated as having a particular cross-sectional shape, the cross-sectional shape illustrated in FIG. 3 merely represents one example cross-sectional shape of many. Similarly, while the components of the urine suction device 300 (e.g., the motor 310, the pump 312, the acoustic silencer 314, the controller 316, and the power supply 318, etc.) are illustrated in respective positions within the housing 308, one or more of the components can be positioned elsewhere within the housing 308 or external to the housing 308. The housing 308 can form a cavity or recess 320 for retaining the container 306. The cavity or recess 320 can have a profile which substantially matches a base portion of the container 306 such that base portion of the container 306 is reliably retained within the recess 320.

The container 306 can include a body 322 and a lid 324. The body 322 can retain or store a quantity of fluid, such as, urine or other fluid which is extracted or pumped from a urine collection device (not shown). The lid 324 can be affixed to the body 322, for example, by a threaded engagement, a friction fit engagement, a protrusion interlocking engagement, or any other mechanism capable of retaining the lid 324 to the body 322. The lid 324 can include one or more inlets 326 which receive the respective tubes 302, 304 or other fluid conduits and place the container 306 in fluid communication with a urine collection device (not shown) and/or the urine suction device 300. The one or more inlets 326 can form respective apertures within the lid 324 to place the container 306 in fluid communication with a urine collection device (not shown) and/or the urine suction device 300. While the one or more inlets 326 are illustrated as being formed on the lid 324, the one or more inlets 326 can additionally, or alternatively, be formed on the body 322 of the container 306.

In some embodiments, the pump 312 can intake fluid (e.g., air) through the tube 302 and exhaust the fluid through a conduit 328. In other words, while the pump 312 is being driven by the motor 310, fluid (e.g., air) can be drawn or sucked into the pump 312 and exhausted through the conduit 328. This process can generate audible acoustic waves which alert persons that urine or other fluid is currently being drawn or extracted from the urine collection device. For example, operation of the pump 312 (e.g., oscillating a diaphragm within the pump 312) and exhausting fluid through the conduit 328 can generate acoustic waves which are detectable by persons located near the urine suction device 300.

The acoustic silencer 314 can be in fluid communication with the conduit 328 and the pump 312. For example, the conduit 328 can be coupled to the pump 312 and the acoustic silencer 314 such that air exhausted from the pump 312 propagates through the conduit and into the acoustic silencer 314. The air exhausted through the conduit 328 can form acoustic waves which have properties (e.g., wavelength, frequency, amplitude, etc.) correlating to operation of the pump 312 and the motor 310.

In some embodiments, the acoustic silencer 314 can include noise dampening materials and/or one or more components which attenuate acoustic waves within the acoustic silencer 314 by redirecting the acoustic waves to generate destructive interference or otherwise muffle the acoustic waves. The one or more components within the acoustic silencer 314 can direct the acoustic waves along the non-linear airflow path which attenuates the acoustic waves as they propagate within the acoustic silencer 314. For example, the acoustic silencer can include multiple internal chambers, one or more conduits, one or more baffles, internal walls, plates, a combination thereof, or any other structure capable of directing acoustic waves along the non-linear airflow path.

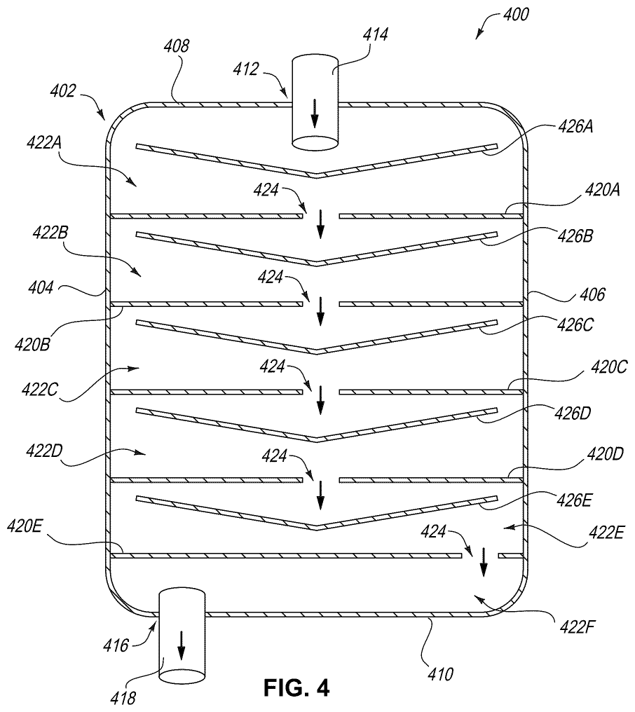

In some embodiments, the acoustic silencer 314 can attenuate or otherwise reduce an amplitude of the acoustic waves by at least 5%, about 5% to about 15%, about 15% to about 30%, about 30% to about 50%, about 50% to about 75%, or more than 75%. The acoustic silencer can attenuate or otherwise reduce one or more acoustic waves having frequencies within a range of about 20 Hz to about 4 kHz. In some embodiments, the one or more acoustic waves can be attenuated or reduced by at least about 1 dB, at least about 5 dB, at least about 10 dB, at least about 15 dB, at least about 20 dB, at least about 25 dB, at least about 30 dB, at least about 35 dB, at least about 40 dB, at least about 45 dB, at least about 50 dB, about 1 dB to about 5 dB, about 2.5 dB to about 7.5 dB, about 5 dB to about 15 dB, about 10 dB to about 20 dB, about 15 dB to about 25 dB, about 20 dB to about 30 dB, about 25 to about 35 dB, about 30 dB to about 40 dB, about 35 dB to about 45 dB, about 40 dB to about 50 dB. The acoustic silencer 114 will be further described below with reference to FIGS. 4-6.

FIG. 4 shows a schematic cross-section view of an acoustic silencer 400 for a urine suction device, according to an example embodiment. The acoustic silencer 400 may be used in any of the urine suction devices disclosed herein and, except as otherwise disclosed herein, may be the same as any of the silencers disclosed herein. The acoustic silencer 400 includes an enclosure 402 having a three-dimensional shape, such as, a cube, a cylinder, a cone, a sphere, an ellipsoid, another three-dimensional geometric shape, or a combination thereof. For example, the enclosure 402 can include a first sidewall 404 and a second sidewall 406. In some embodiments, the first and second sidewalls 404, 406 can form a singular sidewall that wraps around a periphery of the enclosure 402, such as, when the enclosure 402 forms a circular or elliptic cylinder. In some embodiments, the first and second sidewalls 404, 406 can distinct and separate, such as, when the enclosure 402 forms a cube or rectangular cuboid.

In some embodiments, the enclosure 402 can include a third sidewall 408 and a fourth sidewall 410. The third sidewall 408 can include an inlet 412 placing the acoustic silencer 400 in fluid communication with a conduit 414 (e.g., conduit 328). For example, the conduit 414 can be extended through the inlet 412. In some embodiments, the inlet 412 can form a projection which is received within or surrounds a portion of the conduit 414 to place the conduit 414 in fluid communication with the acoustic silencer 400. While the inlet 412 is illustrated as a through-hole or aperture in FIG. 4, the inlet 412 can be any structure or element enabling fluid communication between the acoustic silencer 400 and the conduit 414.

The fourth sidewall 410 can include an outlet 416 placing the acoustic silencer 400 in fluid communication with an external environment (e.g., an internal volume of the housing 308, an environment external to the housing 308, etc.). For example, the outlet 416 can include a conduit 418 that releases any residual acoustic waves from the acoustic silencer 400. While the outlet 416 is illustrated as a conduit 418 in FIG. 4, the outlet 416 can be any structure or element enabling fluid communication between the acoustic silencer 400 and an environment external to the acoustic silencer 400. The enclosure 402 can be manufactured out of any material including, polymers, ceramics, metals, or combinations thereof. The enclosure 402 can be formed using any manufacturing process, such as, molding, machining, stamping, hydroforming, or a combination thereof.

Acoustic waves within the acoustic silencer 400 can propagate or travel along a non-linear airflow path that extends between the conduit 414 and conduit 418 of the acoustic silencer 400. In some embodiments, the non-linear airflow path, as illustrated as directional arrows throughout the acoustic silencer 400, can be a singular path along which acoustic waves are directed to travel within the acoustic silencer 400. For example, the acoustic waves can enter the acoustic silencer through a single inlet (e.g., inlet 412) and exit the acoustic silencer 400 through a single outlet (e.g., outlet 416). However, in some examples, the acoustic waves can enter through more than one inlet and exit through one or more outlets. Additionally, or alternatively, components and/or structures disposed within the acoustic silencer 400 can define a plurality of non-linear airflow paths within the acoustic silencer 400. In other words, while the non-linear airflow path is described as a singular path in some embodiments, the non-linear airflow path can include multiple paths that diverge and/or intersect.

In embodiments, the acoustic silencer 400 can include one or more dividers 420A-E which at least partially separate an internal cavity of the enclosure 402 into chambers 422A-F. Each of the one or more chambers 422A-F can function as an echo chamber wherein the acoustic waves reverberate and destructively interfere to reduce or diminish the amplitude of the acoustic waves. While each of the dividers 420A-E are illustrated as planar or flat, one or more of the dividers 420A-E can be curved or include a bend in some embodiments. Each of the one or more dividers 420A-E can form at least one aperture 424 (e.g., one aperture 424) providing fluid communication between the chambers 422A-F. Each aperture 424 can be formed along the divider 420. For example, the aperture 424 can be disposed on the divider 420E and adjacent the second sidewall 406 of the enclosure 402 while the aperture 424 can be disposed at a mid-section of the divider 420A. Each of the one or more dividers 420A-E can be manufactured out of any material including, polymers, ceramics, metals, or combinations thereof. For example, each of the one or more dividers 420A-E can be metal or plastic sheets which are welded (e.g., sonic welded) or otherwise affixed between the first and second walls 404, 406. Each of the one or more dividers 420A-E can be formed using any manufacturing process, such as, molding, machining, stamping, hydroforming, or a combination thereof.

In embodiments, the acoustic silencer 400 can include one or more baffles or structures 426A-E (e.g., one, at least one, two or more, three or more, etc.) that direct or guide acoustic waves along the non-linear airflow path. Each of the one or more baffles 426A-E can include planar surfaces, curved surfaces, angled surfaces, perforated surfaces, slotted surfaces, or combinations thereof. For example, each of the baffles 426A-E can be shaped or formed to resemble a “V” or planar portions angled relative to one another such that one or more acoustic waves propagating along the non-linear airflow path (e.g., through an aperture 424) are reverberated or reflected to cause destructive interference.

In embodiments, one or more acoustic waves can propagate out of the inlet 412 and reflect off of the baffle 426A. The one or more acoustic waves can continue to repeatedly reflect between the baffle 426A and the third sidewall 408 until the one or more acoustic waves propagate through gap regions 428 between the baffle 426A and the enclosure 402. Subsequently, at least a portion of the one or more acoustic waves can repeatedly reflect between the baffle 426A and the divider 420A until the one or more acoustic waves propagate through the aperture 424 formed in the divider 420A. This process can continue as the one or more acoustic waves propagate through the remaining chambers 422B-F and out of the outlet 416.

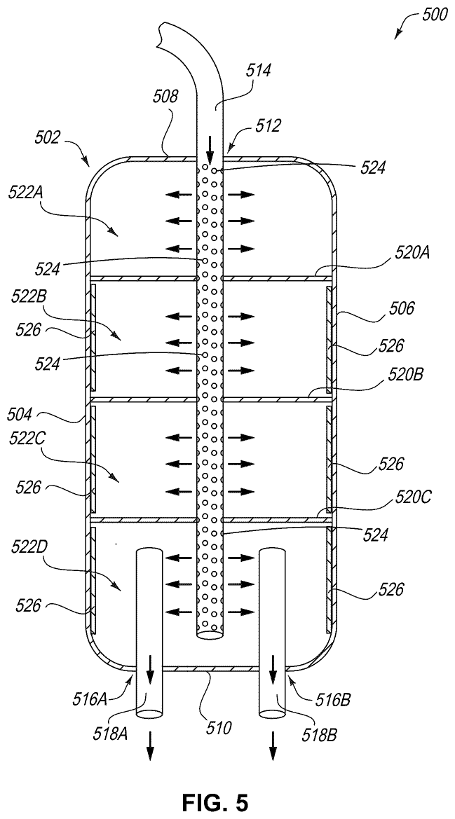

FIG. 5 shows a schematic cross-section view of an acoustic silencer 500 for a urine suction device, according to another example embodiment. The acoustic silencer 500 may be used in any of the urine suction devices disclosed herein and, except as otherwise disclosed herein, may be the same as any of the silencers disclosed herein. The acoustic silencer 500 includes an enclosure 502 having a three-dimensional shape, such as, a cube, a cylinder, a cone, a sphere, an ellipsoid, another three-dimensional geometric shape, or a combination thereof. The enclosure 502 can be substantially similar to and can include some or all of the features of the enclosure 402. For example, the enclosure 502 can include a first sidewall 504, a second sidewall 506, a third sidewall 508, and a fourth sidewall 510. The third sidewall 508 can include an inlet 512 placing the acoustic silencer 500 in fluid communication with a conduit 514 (e.g., conduit 328). The fourth sidewall 510 can include one or more outlets 516A, 516B placing the acoustic silencer 500 in fluid communication with an external environment (e.g., an internal volume of the housing 308, an environment external to the housing 308, etc.). Acoustic waves within the acoustic silencer 500 can propagate or travel along a non-linear airflow path, as illustrated as directional arrows throughout the acoustic silencer 500, that extends between the inlet 512 and the one or more outlets 516 of the acoustic silencer 500. The outlet 516 can include conduits 518A, 518B that release any residual acoustic waves from the acoustic silencer 500.

In some embodiments, the acoustic silencer 500 can include one or more dividers 520A-C which separate an internal cavity of the enclosure 502 into chambers 522A-D. Each of the one or more chambers 522A-E can function as an echo chamber wherein the acoustic waves reverberate and destructively interfere to reduce or diminish the amplitude of the acoustic waves. The conduit 514 can extend into the enclosure 502 and be in fluid communication with the chambers 522A-D. For example, as shown in FIG. 5, the conduit 514 can extend through each of the dividers 520A-C and include perforations or through-holes 524 that enable one or more acoustic waves to propagate between the conduit 514 and each of the chambers 522A-D. While perforation or through-holes 524 are illustrated in FIG. 5, the conduit 514 can alternatively, or additionally, include one or more slots or any other opening having a different geometry than illustrated that places the conduit 514 in fluid communication with one or more of the chambers 522A-D.

The acoustic silencer 500 can include a material 526 disposed within the enclosure 502, for example, the material 526 can be positioned adjacent at least one of the first, second, third, or fourth sidewalls 504, 506, 508, 510. The material 526 can dampen or attenuate at least a portion of the one or more acoustic waves that propagate within the enclosure 502. In some embodiments, the material 526 can include at least one of a foam (e.g., open-cell foam), a spun-fiber (e.g., mineral wool), or a polyester. The material 526 can be adhered, fastened, molded, or otherwise affixed to any surface within the enclosure 502, such as, the first, second, third, or fourth sidewalls 504, 506, 508, 510, the conduit 514, the one or more dividers 520A-C, the one or more outlets 516A, 516B, or a combination thereof.

In some embodiments, the non-linear airflow path can be a singular path along which acoustic waves are directed to travel within the acoustic silencer 500. For example, the acoustic waves can enter the acoustic silencer through a single inlet (e.g., inlet 512) and exit the acoustic silencer 500 through a single outlet (e.g., one of outlets 516A, 516B). However, in some examples, the acoustic waves can enter through more than one inlet and exit through one or more outlets. Additionally, or alternatively, components and/or structures disposed within the acoustic silencer 500 can define a plurality of non-linear airflow paths within the acoustic silencer 500. In other words, while the non-linear airflow path is described as a singular path in some embodiments, the non-linear airflow path can include multiple paths that diverge and/or intersect.

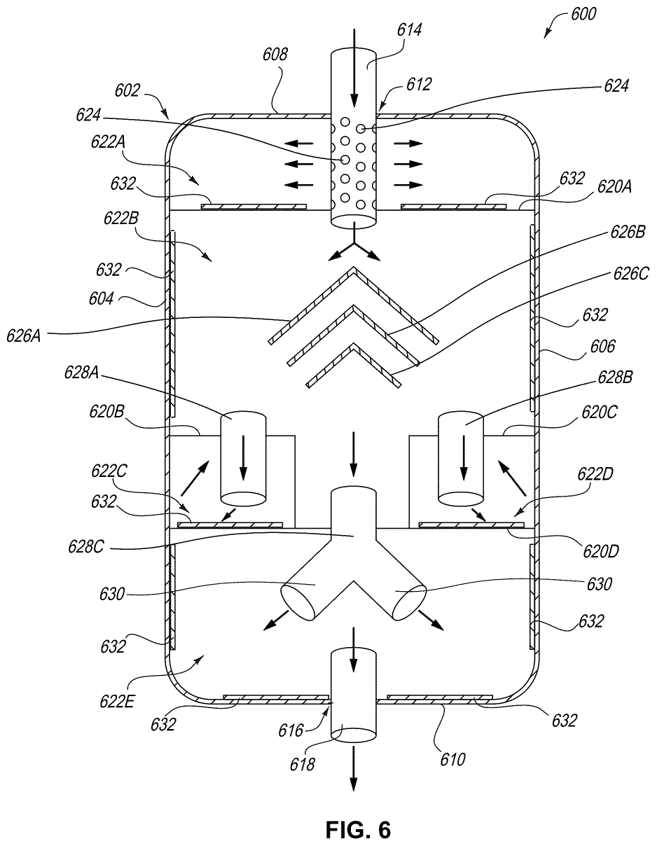

FIG. 6 shows a schematic cross-section view of an acoustic silencer 600 for a urine suction device, according to yet another example embodiment. The acoustic silencer 600 may be used in any of the urine suction devices disclosed herein and, except as otherwise disclosed herein, may be the same as any of the silencers disclosed herein. The acoustic silencer 600 includes an enclosure 602 having a three-dimensional shape, such as, a cube, a cylinder, a cone, a sphere, an ellipsoid, another three-dimensional geometric shape, or a combination thereof. The enclosure 602 can be substantially similar to and can include some or all of the features of the enclosures 402, 502. For example, the enclosure 602 can include a first sidewall 604, a second sidewall 606, a third sidewall 608, and a fourth sidewall 610. The third sidewall 608 can include an inlet 612 placing the acoustic silencer 600 in fluid communication with a conduit 614 (e.g., conduit 328). The fourth sidewall 610 can include an outlet 616 and a conduit 618 placing the acoustic silencer 600 in fluid communication with an external environment (e.g., an internal volume of the housing 308, an environment external to the housing 308, etc.). Acoustic waves within the acoustic silencer 600 can propagate or travel along a non-linear airflow path that extends between the conduit 614 and the conduit 618 of the acoustic silencer 600.

In some embodiments, the acoustic silencer 600 can include one or more dividers 620A-D which separate an internal cavity of the enclosure 602 into chambers 622A-E. Each of the one or more chambers 622A-E can function as an echo chamber wherein the acoustic waves reverberate and destructively interfere to reduce or diminish the amplitude of the acoustic waves. The conduit 614 can extend into the enclosure 602 and can be directly or indirectly in fluid communication with the chambers 622A-E. For example, as shown in FIG. 6, the conduit 614 can extend through the dividers 620A and include perforations or through-holes 624 that enable one or more acoustic waves to propagate between the conduit 614 and each of the chambers 622A, 622B. While perforation or through-holes 624 are illustrated in FIG. 6, the conduit 614 can alternatively, or additionally, include one or more slots or any other opening that places the conduit 614 in fluid communication with one or more of the chambers 622A-E.

The acoustic silencer 600 can include one or more components or structures within the enclosure 602 that can direct or redirect propagation of the acoustic waves, for example, the acoustic silencer 600 can include one or more baffles 626A-C. The baffles 626A-C can be substantially similar to and can include some or all of the features of the baffles 426A-E. For example, each of the baffles 626A-C can include planar surfaces, curved surfaces, angled surfaces, perforated surfaces, slotted surfaces, or combinations thereof. Each of the baffles 626A-C can be stacked or positioned adjacent one another such that acoustics waves repeatedly reflected between surfaces of the baffles 626A-C.

In some embodiments, the acoustic silencer 600 can include one or more internal conduits 628A, 628B that direct acoustic waves into the partially enclosed portions of the enclosure 602. For example, the internal conduit 628A can direct one or more acoustic waves into the chamber 622C. The one or more acoustic waves can reflect and destructively interfere with one another while within the chamber 622C and while entering/exiting the chamber 622C through the internal conduit 628A. The internal conduit 628B can direct one or more acoustic waves into the chamber 622D. The one or more acoustic waves can reflect and destructively interfere with one another while within the chamber 622D and while entering/exiting the chamber 622D through the internal conduit 628B. In some embodiments, one or both of the internal conduits 628A, 628B can include perforation or through-holes along at least a partial length of the internal conduits 628A, 628B.

In embodiments, the acoustic silencer 600 can include an internal conduit 628C that forms multiple paths for the one or more acoustic waves to propagate. For example, the internal conduit 628C can include multiple passages 630 that direct the one or more acoustic waves to propagate to either side of the outlet 616 such that the acoustic waves are forced to propagate within the chamber 622E before exiting the enclosure 602 through the outlet 616.

The acoustic silencer 600 can include a material 632 disposed within the enclosure 602, for example, the material 632 can be positioned adjacent at least one of the first, second, third, or fourth sidewalls 604, 606, 608, 610. The material 632 can dampen or attenuate at least a portion of the one or more acoustic waves that propagate within the enclosure 602. In some embodiments, the material 632 can include at least one of a foam (e.g., open-cell foam), a spun-fiber (e.g., mineral wool), or a polyester. The material 632 can be adhered, fastened, molded, or otherwise affixed to any surface within the enclosure 602, such as, the first, second, third, or fourth sidewalls 604, 606, 608, 610, the conduit 614, the one or more dividers 620A-D, the one or more internal conduits 628A, 628B, 628C, the outlet 616, or a combination thereof.

In some embodiments, the non-linear airflow path can be a singular path along which acoustic waves are directed to travel within the acoustic silencer 600. For example, the acoustic waves can enter the acoustic silencer through a single inlet (e.g., inlet 612) and exit the acoustic silencer 600 through a single outlet (e.g., outlet 616). However, in some examples, the acoustic waves can enter through more than one inlet and exit through one or more outlets. Additionally, or alternatively, components and/or structures disposed within the acoustic silencer 600 can define a plurality of non-linear airflow paths within the acoustic silencer 600. In other words, while the non-linear airflow path is described as a singular path in some embodiments, the non-linear airflow path can include multiple paths that diverge and/or intersect.

FIGS. 4-6 illustrate example embodiments of acoustic silencers having one or more structures, components, and/or features. While various embodiments of the acoustic silencers have been described above, it should be understood that they have been presented by way of example only, and not limitation. Various changes in form and detail may be made. For example, although various embodiments have been described as having particular features and/or combinations of components, other embodiments are possible having any combination or sub-combination of any features and/or components from any of the embodiments described herein. In addition, the specific configurations of the various components can also be varied. For example, the size and specific shape of the various components can be different than the embodiments shown, while still providing the functions as described herein.

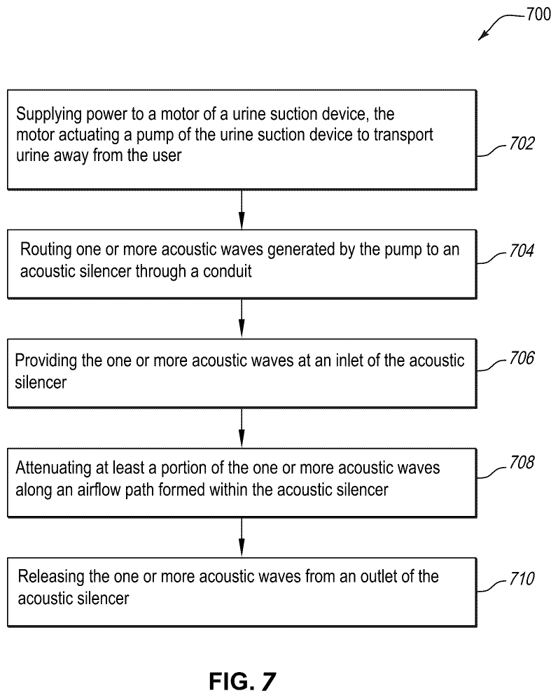

FIG. 7 illustrates an example method 700 for reducing noise pollution of a urine suction device. The suction device can be substantially similar to, and can include some or all of the features and/or components of the suction devices described herein, such as suction devices 100, 200, 300. For example, the suction device can include a motor, a pump, one or more speakers, a controller, a power supply, one or more sensors, and/or any other component of the other suction devices disclosed herein. The acoustic silencer can be substantially similar to, and can include some or all of the features and/or components of the acoustic silencers described herein, such as acoustic silencers 400, 500, 600. For example, the acoustic silencer can include one or more inlets, one or more outlets, one or more conduits, one or more baffles, one or more dividers, one or more chambers, a material that dampens acoustic waves, or a combination thereof.

The method 700 includes the act 702 of supplying power (e.g., electrical power, pneumatic power, etc.) to a motor of a urine suction device, the motor actuating a pump of the urine suction device to transport urine away from the user. The method 700 includes the act 704 of routing one or more acoustic waves generated by the pump to an acoustic silencer through a conduit. The method 700 includes the act 706 of providing the one or more acoustic waves at an inlet of the acoustic silencer. The method 700 can include the act 708 of attenuating at least a portion of the one or more acoustic waves along an airflow path formed within the acoustic silencer. The method 700 can include the act 710 of releasing the one or more acoustic waves from an outlet of the acoustic silencer.

Accordingly, the method 700 can be utilized to attenuate or reduce noise generated by operation of the motor and/or pump within the suction device. The method 700 can include more acts than the acts 702-710.

The method 700 includes the act 702 of supplying power to a motor of a urine suction device, the motor actuating a pump of the urine suction device to transport urine away from the user. In some embodiments, the power can be supplied directly to the motor as electrical power from a power supply. Alternatively, or additionally, the electrical power can be supplied to the motor through a controller coupled to the power supply. In some embodiments, the motor can be driven by air, such as, compressed air supplied to the motor. The motor can be substantially similar to, and can include some or all of the features and/or components of the motors described herein, such as motor 110, 310. For example, the motor can operate on alternating current (AC) or direct current (DC) and can be a brushless motor or a brushed motor. Additionally, or alternatively, the motor can be DC series motor, a DC shunt motor, a stepper motor, a linear motor, a servo motor, a combination thereof, or any other motor capable of the functionality described herein. Similarly, the pump can be substantially similar to, and can include some or all of the features and/or components of the pumps described herein, such as pumps 112, 312. For example, the pump can be a rotary lobe or gear pump, a diaphragm pump, a piston, a screw pump, a combination thereof, or any other pump capable of the functionality described herein.

The method 700 includes the act 704 of routing one or more acoustic waves generated by the pump to an acoustic silencer through a conduit. The method 700 includes the act 706 of providing the one or more acoustic waves at an inlet of the acoustic silencer. In embodiments, the conduit can extend from the pump and through the inlet of the acoustic silencer. The inlet can form a projection which is received within or surrounds a portion of the conduit to place the conduit in fluid communication with the acoustic silencer. The inlet can be any structure or element enabling fluid communication between the acoustic silencer and the conduit.

The method 700 can include the act 708 of attenuating at least a portion of the one or more acoustic waves along an airflow path formed within the acoustic silencer. In some embodiments, the acoustic silencer can include an enclosure and one or more components which attenuate at least a portion of acoustic waves that propagate into the enclosure. For example, the acoustic silencer can include noise dampening materials, such as, foam, mineral wool, polyester, and/or any other material known to dampen or attenuate acoustic waves. Additionally, or alternatively, the acoustic silencer can include one or more components which attenuate acoustic waves within the enclosure by redirecting the acoustic waves to generate destructive interference. In other words, one or more components or structures within the acoustic silencer can direct the acoustic waves along the airflow path which attenuates the acoustic waves as they propagate within the acoustic silencer. For example, the acoustic silencer can include multiple internal chambers, one or more conduits, one or more baffles, internal walls, plates, a combination thereof, or any other element capable of directing acoustic waves along the airflow path.

In some embodiments, the method 700 can include the act 710 of releasing the one or more acoustic waves from an outlet of the acoustic silencer. The outlet can place the acoustic silencer in fluid communication with an external environment (e.g., an internal volume of the housing 308, an environment external to the housing 308, etc.). For example, the outlet can release any residual acoustic waves from the acoustic silencer. The outlet 416 can be any structure or element enabling fluid communication between the acoustic silencer and an environment external to the acoustic silencer.

While various embodiments of the urine suction systems and devices have been described above, it should be understood that they have been presented by way of example only, and not limitation. Those of ordinary skill in the art having the benefit of this disclosure would recognize that the ordering of certain steps may be modified and such modifications are in accordance with the variations of the invention. Additionally, certain of the steps may be performed concurrently in a parallel method when possible, as well as performed sequentially as described above. The embodiments have been particularly shown and described, but it will be understood that various changes in form and details may be made.

For example, although various embodiments have been described as having particular features and/or combinations of components, other embodiments are possible having any combination or sub-combination of any features and/or components from any of the embodiments described herein. In addition, the specific configurations of the various components can also be varied. For example, the size and specific shape of the various components can be different than the embodiments shown, while still providing the functions as described herein.

1. (canceled)

2. A urine suction device, comprising:
a motor;
a pump configured to be driven by the motor, the pump being configured to extract urine from a urine collection device;
one or more conduits coupled to the pump and configured to exhaust a fluid from the pump; and
an acoustic silencer in fluid communication with the one or more conduits and the pump, the acoustic silencer including an enclosure defining an internal cavity, an enclosure inlet, and an enclosure outlet, the acoustic silencer defining a non-linear airflow path between the inlet and the outlet of the enclosure;
wherein the one or more conduits includes an outlet conduit (“OC”) extending through the outlet into the internal cavity.

3. The urine suction device of claim 2, wherein:
the one or more conduits includes an inlet conduit (“IC”) extending through the enclosure inlet, the IC including an IC inlet and an IC outlet opposite the IC inlet, the IC inlet disposed in the internal cavity and the IC outlet spaced from the internal cavity;
the OC includes an OC inlet and an OC outlet, the OC inlet disposed in the internal cavity and the OC outlet of spaced from the internal cavity; and
the IC outlet is positioned closer to the enclosure outlet than the OC inlet.

4. The urine suction device of claim 2, wherein:
the enclosure defines an additional enclosure outlet; and
the one or more conduits includes an additional outlet conduit extending through the additional enclosure outlet into the internal cavity.

5. The urine suction device of claim 2, wherein the enclosure include a sidewall that defines the outlet, the sidewall including an internal surface that defines the internal cavity; and
wherein the acoustic silencer further includes a material disposed on the internal surface of the sidewall, the material configured to dampen or attenuate at least a portion of one or more acoustic waves propagating within the internal cavity.

6. The urine suction device of claim 2, further comprising a housing defining an interior volume, the housing defining a recess configured to receive a container, the housing not configured to cover a top of the container, the recess distinct and spaced from the interior volume.

7. The urine suction device of claim 6, wherein the motor, the pump, the one or more conduits, and the acoustic silencer are disposed in the interior volume.

8. The urine suction device of claim 2, wherein the acoustic silencer includes one or more dividers separating the enclosure into two or more chambers, the one or more dividers defining at least one aperture providing fluid communication between the two or more chamber.

9. The urine suction device of claim 8, wherein the one or more conduits include an inlet conduit extending through the inlet.

10. The urine suction device of claim 8, wherein a majority of each of the two or more chambers is open space.

11. The urine suction device of claim 8, wherein the one or more dividers include a plurality of dividers, and the aperture of one of the plurality of dividers is closer to a side wall of the enclosure than the aperture of another one of the plurality of dividers, wherein the side wall does not define at least one inlet and at least one outlet of the enclosure.

12. The urine suction device of claim 11, wherein each of the plurality of dividers extend from one side wall of the enclosure to an opposing side wall of the enclosure.

13. The urine suction device of claim 8, wherein the one or more dividers defining at least one of the two or more chambers defines a single aperture, and wherein the at least one of the two or more chambers is only in fluid communication with the inlet, the outlet, and a remainder of the two or more chambers via the single aperture.

14. The urine suction device of claim 8, wherein the acoustic silencer further includes one or more baffles disposed in one or more of the two or more chambers, the baffles spaced from the one or more conduits and defining a gap between the one or more baffles and the enclosure.

15. The urine suction device of claim 8, wherein the acoustic silencer includes at least one branched internal conduit distinct and separate from the one or more conduits, the at least one branched internal conduit in fluid communication with a plurality of the two or more chambers of the enclosure, the at least one branched internal conduit is branched downstream of an inlet of the at least one branched internal conduit to form multiple paths through the at least one branched internal conduit.

16. The urine suction device of claim 8, wherein the inlet conduits includes a portion extending through the inlet, into the enclosure, and into a plurality of the two or more chamber, the portion of the inlet conduit defines one or more perforations in a side wall thereof, the one or more perforations in the fluid communication with the plurality of the two or more chamber.

17. The urine suction device of claim 8, wherein the OC includes an OC inlet and an OC outlet, the OC inlet disposed in the internal cavity and the OC outlet of spaced from the internal cavity, the OC inlet positioned closer to the one or more dividers than the outlet.

18. The urine suction device of claim 8, wherein a flow path of the OC is not aligned with a flow path through the one or more dividers.

19. The urine suction device of claim 1, wherein at least a portion of the one or more conduits that extend into the internal cavity defines a plurality of perforations.

20. A system for transporting urine away from a user, the system comprising:
a urine collection device;
a container; and
a urine suction device coupled to the urine collection device and configured to draw urine from the urine collection device into the container, the urine suction device including:
a motor;
a pump configured to be driven by the motor, the pump being configured to extract urine from a urine collection device;
one or more conduits coupled to the pump and configured to exhaust a fluid from the pump; and
an acoustic silencer in fluid communication with the one or more conduits and the pump, the acoustic silencer including an enclosure defining an internal cavity, an enclosure inlet, and an enclosure outlet, the acoustic silencer defining a non-linear airflow path between the inlet and the outlet of the enclosure;
wherein the one or more conduits includes an outlet conduit extending through the outlet into the internal cavity.

21. A method for reducing noise pollution of a urine suction device with an acoustic silencer, the method comprising:
supplying power to a motor of the urine suction device, the motor actuating a pump of the urine suction device to transport urine away from a user;
routing one or more acoustic waves generated by the pump to the acoustic silencer through one or more conduits, the one or more conduits coupled to the pump and configured to exhaust a fluid from the pump, the acoustic silencer including an enclosure defining an internal cavity, an enclosure inlet, and an enclosure outlet, the acoustic silencer defining a non-linear airflow path between the inlet and the outlet of the enclosure;
wherein the one or more conduits includes an outlet conduit extending through the outlet into the internal cavity attenuating at least a portion of the one or more acoustic waves along the airflow path formed within the acoustic silencer; and
releasing the one or more acoustic waves from the outlet conduit.
